(19) United States
(12) Plant Patent Application Publication
(10) Pub. No.: US 2018/0249612 P1
(43) Pub. Date: Aug. 30, 2018

(54) Sweet cherry tree named 'IFG Cher-Five'

(50) ;

(71) Applicant: David Cain, Bakersfield, CA (US)

(72) Inventor: David Cain, Bakersfield, CA (US)

(21) Appl. No.: 15/530,772

(22) Filed: Feb. 28, 2017

(51) Int. Cl. A01H 6/74

(52) U.S. Cl. PLT/181

(57) ABSTRACT

This invention is a new and distinct sweet cherry tree variety denominated ‘IFG Cher-five’. The new sweet cherry tree is characterized by producing large size dark red fruits having flat-round shape. Fruits ripen early about zero to two days ahead of the ‘Brooks’ variety (U.S. Plant Pat. No. 6,676). The ‘IFG Cher-five’ has firm, medium acid fruit with an excellent cherry flavor. Fruits are more tolerant of rain induced cracking, and high temperature induced doubling than ‘Brooks’ and stores better than ‘Brooks’.

Latin name of the genus and species claimed: Prunus avium.

Variety denomination: ‘IFG Cher-five’.

### BACKGROUND OF THE INVENTION

The new and distinct sweet cherry tree described and claimed herein originated from open pollinated seeds of fruits of an early ripening unidentified variety growing in a 12 commercial orchard located near Bakersfield, in Kern County, Calif. collected in May 2001. The seeds were stratified, germinated and the resulting one seedling was planted in the field near Delano, Kern County, Calif. in April 2002. The present variety of sweet cherry tree was selected as a single plant in May 2005 and was first asexually propagated in January 2006 by grafting onto Prunus mahalab rootstock. This propagule was found to reproduce true-to-type by asexual propagation. All propagation was done near Delano, Kern County Calif.

### BRIEF SUMMARY OF THE INVENTION

Sweet cherries have traditionally been grown in climates with long cold winters and cool to moderately warm summers. Such climates provide enough cold winter temperatures to allow normal growth to resume in the spring and summer temperatures that are low enough not to induce production of unmarketable double or spurred fruit, but it limits the seasonality that cherries are available. The sweet cherry breeding program focuses on developing types of cherries that will grow in regions with low winter chilling and high summer temperatures so that the fruit will ripen before fruit in traditional growing regions.

The new sweet cherry tree ‘IFG Cher-five’ is characterized by producing large size dark red fruits having flat-round shape. Fruits ripen early about zero to two days ahead of the ‘Brooks’ variety (U.S. Plant Pat. No. 6,676). The ‘IFG Cher-five’ has firm, medium acid fruit with an excellent cherry flavor. Fruits are more tolerant of rain induced cracking than ‘Brooks’. The tree has a medium-low chilling requirement, slightly lower than the ‘Brooks’ variety. It produces fewer doubled and spurred fruits as compared to the ‘Brooks’ variety in high summer temperature regions such as the Southern San Joaquin Valley of California. Fruits of ‘IFG Cher-five’ have medium long, thick stems that remain attached and stay green during storage and shipping. It has more desirable darker red skin and flesh than the ‘Brooks’ variety and exhibits less pitting and stores better than the ‘Brooks’ variety.

In comparison with the ‘Glenred’ (U.S. Plant Pat. No. 12,859), the ‘IFG Cher-five’ ripens about two to four days earlier. The fruit stems of ‘IFG Cher-five’ are substantially shorter than ‘Glenred’ and remain attached better during storage and shipping.

### BRIEF DESCRIPTION OF THE DRAWING

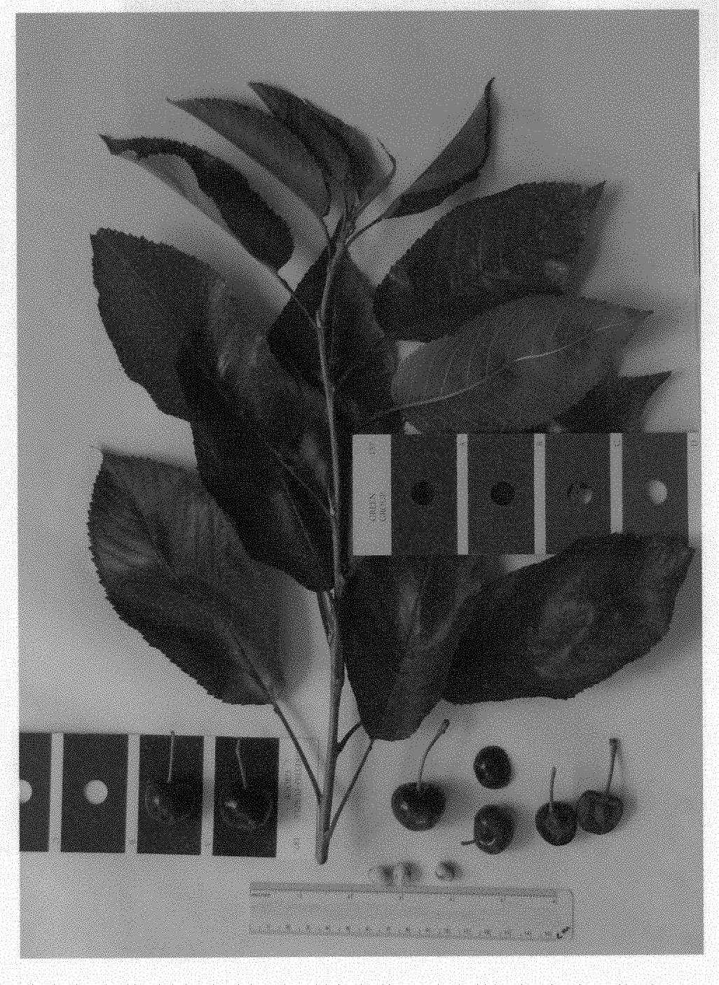

The accompanying photographic drawing in FIG. 1 illustrates in full color ‘IFG Cher-five’. The photograph was taken outdoors with indirect lighting. The colors are as nearly true as is reasonably possible in a color representation of this type. An actively growing shoot tip collected at harvest can be seen in the upper portion of the drawing. Typical mature fruit and fruit in cross-section and cleaned and dried fruit pits are displayed on the lower half of the drawing.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published in 2001 by The Royal Horticultural Society, London, England.

Throughout this specification subjective description values conform to those set forth by the International Board for Plant Genetic Resources (IBPGR) ‘Cherry Descriptor List’ (Prunus spp.) (1985) which was developed with full support from the Commission of the European Communities (CEC) Programme Committee for Plant Disease Resistance Breeding and the Use of Genebanks.

The descriptive matter which follows pertains to ‘IFG Cher-five’ plants grown in the vicinity of Delano, Kern County, Calif. during 2013 and 2014, and is believed to apply to plants of the variety grown under similar conditions of soil and climate elsewhere:
Tree:
   General.—Age — 13 yrs. old Height — About 3.1 M when pruned Width — About 2.6 M when pruned Vigor — Medium Density of foliage — Dense Form — Upright Rootstock — Prunus mahaleb Resistance to — Insects: Average typical of Prunus avium species. Diseases: Average typical of Prunus avium species. Chilling requirements — Medium, slightly lower than the ‘Brooks’ variety Graft compatibility — Good; produces compatible graft unions with Prunus avium, ‘Mazzard’ seedlings (non-patented) and Prunus mahaleb seedlings (non-patented).
   Trunk.—Trunk diameter of 13 year old trees, 30 cm above the soil line — About 20.1 cm Lenticel size — Large Lenticel dimensions — Length: About 1.2 cm. Width: About 0.4 cm Lenticel shape — Elliptical shape oriented horizontally Lenticel color — Grey: 201C Trunk surface texture — Medium to slightly rough Outer bark color — The following colors were observed: Grey: 201C and Greyed-orange: 176B.
Branches:
   1 year old wood.—Vertical top growth length — About 31.2 cm Horizontal Growth Length — About 37.6 cm Diameter — Vertical growth: About 1.3 cm. Horizontal Growth: About 0.8 cm Internode length — About 3.9 cm Number of lenticels — Few Lenticel size — Small Lenticel dimensions — Length: About 0.1cm. Width: About 0.1 cm Lenticel shape — Round Bark color — The following colors were observed: Greyed-orange; 166A and 166B.
   2 year old wood.—Length — About 54 cm Diameter — About 1.4 cm Internode length — About 5.1 cm Number of lenticels — Medium Lenticel dimensions — Length: About 0.1 cm. Width: About 0.2 cm Lenticel shape — Elliptical shape oriented horizontally Number of fruiting spurs on 2-year wood — 4 to 12, Average: 7 Bark color — The following colors were observed: Greyed-orange: 174A and 177B.
Buds:
   Vegetative buds.—Shape — Elongated Vegetative bud dimensions — Length: About 1.0 cm. Width: About 0.4 cm Vegetative bud burst — About Feb. 27, 2014.
   Flower buds.—Flower bud dimensions — Length: About 1.0 cm. Width: About 0.5 cm Shape — Oval Placement — At bud positions 1 to 8 on 1-year wood Average number of flower buds on first year wood — About 8 Number of flower buds per spur on second year wood — 2 to 3, Average: About 3 Color — The following colors were observed: Greyed-orange: 166A and 177A Flower bud burst — About Mar. 1, 2014.
Leaves:
   Mature leaves.—Leaf dimensions — Length: About 12.1 cm. Width: About 5.0 cm Leaf shape — Lanceolate: symmetric on both sides of central axis Shape of tip — Acuminate: broadly Shape of base — Attenuate Margin — Serrated: regular, rounded Surface texture — Smooth Leaf profile — Involute.
   Upper surface.—Upper surface pubescence — None Upper leaf surface color — Green: 139A.
   Lower surface.—Lower surface pubescence — Very sparse Lower leaf surface color — Yellow-green: 147B.
   Petiole.—Petiole dimensions — Length: About 4.8 cm. Width: About 1.6 mm Upper surface of petiole color — Yellow-green: 144A Lower surface of petiole color — Yellow-green: 144B Petiole groove — Medium Petiole pubescence — Very sparse; only on upper surface Venation — Arcuate Vein color — Yellow-Green: 145A.
   Glands.—Number of glands — 1 to 2 Gland dimensions — Length: About 0.2 cm. Width: About 1.2 cm Gland shape — Globose Gland location — On petiole Gland color — Red-purple: 60A Leaf stipule — Not present.
Flowers:
   Blooming period.—Early.
   Blooming dates.—First bloom Mar. 4, 2014, Full bloom Mar. 17, 2014.
   Number of flowers per cluster—2-3. Average: about 2.
   Corolla.—Composed of unfused petals, somewhat overlapping.
   Corolla diameter.—About 3.2 cm.
   Petal number.—5.
   Petal length.—About 1.4 cm.
   Petal width.—About 1.4 cm.
   Margin waviness.—Strong.
   Division of upper margin.—Entire.
   Color of petal upper surface.—White: 155D.
   Color of petal lower surface.—White: 155C.
   Peduncle.—Length: About 1.9 cm. Width: About 0.1 cm.
   Peduncle color.—Yellow-green: 144B.
   Number of sepals.—5.
   Sepal length.—About 0.7 cm.
   Sepal width.—About 0.5 cm.
   Sepal shape.—Triangular.
   Sepal color.—Upper surface: Yellow-green: 144A. Lower surface: Red-purple: 59C.
   Filament.—Length: About 0.5-1.5 cm. Width: About 0.3 mm.
   Filament color.—White: N155C.
   Anther color.—Yellow-orange: 22D.
   Pollen color.—Greyed-orange: 163A.
   Pollen production.—Medium.
   Self-compatibility of flowers.—Self-incompatible.
   Flower compatibility group.—S1S new unidentified allele.
Fruit:
   General.—Ripening period — Early: Approximately: Apr. 25-30, 2014. Use — Fresh market Keeping quality — Good: slightly better than the ‘Brooks’ variety % Titratable acidity — About 0.82% Refractometer test — Soluble solids: About 20.2 brix Firmtech II (g/mm) — About 280 to 339 Flavor — Excellent, good sugar/acid balance with pronounced cherry flavor Juice color — Greyed-purple: 187B Juice amount — Juicy Eating quality — Excellent.
   Stem.—Stem — Length: About 2.6 cm. Width: About 0.2 cm Stem color — The following colors were observed: Yellow-green: 143C and 144B Stem cavity — Medium Stem retention during storage — Very good Stem storage quality — Good.
   Berry.—Uniformity of size — Uniform Shape — Flat-round Fruit Weight — About 10.6 gm Apical Diameter — About 2.5 cm Diameter transversely across suture — About 2.8 cm Diameter at right angle to suture plane — About 2.3 cm Suture — None Percent of excessively deep or split sutures — 0% Doubles — 0%.
   Skin.—Thickness — Medium Texture — Mostly smooth, with very little indentation noted at lenticels Skin color — Greyed-purple: 187B Tendency to tip crack — Not susceptible Tendency to stem cavity crack — Not susceptible.
   Flesh.—Texture — Firm Color — The following colors were observed: Greyed-purple: 187A, and Red: 45C and 45D and 50A.
   Stone.—Shape — Ovate Length: About 1.3 cm Width transversely across suture — About 1.0 cm Width at right angle to suture plane — About 1.1 cm Type — Semi-cling Surface texture — Slightly rough Stone Color when dry — Yellow-white: 158A Tendency to split — None Base — Broadly pointed Apex — Acutely pointed Ventral edge — Prominent narrow suture extending from the base to the midpoint of the stone subtended by 2 minor ridges converging at the base and the apex Dorsal edge — Narrow ridge from base to apex.

What is claimed:

1. A new and distinct variety of sweet cherry tree as herein illustrated and described.
